(19) United States
(12) Patent Application Publication
(10) Pub. No.: US 2026/0249529 A1
(43) Pub. Date: Aug. 27, 2026

(54) PREPARATION METHOD FOR HYDROGEL ELECTRODE

(71) Applicant: DALIAN UNIVERSITY OF TECHNOLOGY, Dalian City (CN)

(72) Inventors: Jiaqi LIN, Dalian City (CN); Hailing XUE, Dalian City (CN); Dongyang WANG, Dalian City (CN)

(73) Assignee: DALIAN UNIVERSITY OF TECHNOLOGY, Dalian City, LN (CN)

(21) Appl. No.: 18/704,053

(22) PCT Filed: Jan. 29, 2024
(86) PCT No.: PCT/CN2024/074511

§ 371 Date: Apr. 21, 2026

(30) Foreign Application Priority Data
Feb. 15, 2023 (CN) 202310117215.0

(51) Int. Cl. B29C 45/14; A61B 5/291; B29K 105/00; B29K 705/14; B29L 31/34

(52) U.S. Cl. CPC B29C 45/14311; A61B 5/291; B29C 45/14336; B29C 45/14639; B29C 2045/14885; B29K 2105/0061; B29K 2705/14; B29L 2031/34

(57) ABSTRACT

The invention presents a method for preparing a hydrogel electrode, involving the following steps: (1) preparing a mold for molding the hydrogel electrode; (2) cleaning an electrode support with isopropanol, ethanol, and deionized water, then drying and treating it with a plasma surface processor; (3) assembling and securing the electrode support and a Ag/AgCl button to the mold; (4) injecting adhesive and conductive hydrogel prepolymer solutions into the mold sequentially. This method ensures the conductivity and durability of the conductive hydrogel, facilitating long-term and repeated use. The adhesive hydrogel forms chemical bonds with the conductive layer and electrode support, preventing issues like interface delamination and friction between the hydrogel and support that often occur with semi-dry electrodes, thus enhancing reliability during use.

### TECHNICAL FIELD

The invention relates to the field of biosensor technology, in particular to a preparation method for a hydrogel electrode.

### BACKGROUND ART

Non-invasive brain-computer interface (BCI) has shown great potential in applications such as sleep monitoring, fatigue alarm, and neural feedback training. Compared with invasive BCI, non-invasive BCI does not pose any security risks to users. The electrodes of traditional non-invasive brain-computer interface for collecting electroencephalogram (EEG) are usually divided into two types: dry electrodes and wet electrodes. Dry electrodes are composed of solid conductive materials, comprising metals, conductive silica gel, and conductive polymers, which can record physiological electrical signals by contacting the skin. However, the dry electrodes increase the skin contact impedance and reduce the stability of the impedance, so the dry electrodes are more sensitive to motion artifacts. In addition, dry electrodes also have other shortcomings, such as insufficient contact with the scalp during use, increased signal noise, and severe distortion of EEG signals due to skin sweating and sweat corrosion. Some dry electrodes (such as metal needle electrodes) may damage the head surface. Wet electrodes mainly comprise conductive paste and silver/silver chloride (Ag/AgCl), this material has the advantages of high exchange current density, low polarization, and constant potential. As a conductive medium, the conductive paste can form an ion channel between the electrode and the skin and ensure that the signal baseline remains smooth throughout the EEG recording process. However, the setting process of wet electrodes using conductive paste is usually very complicated and time-consuming, and it is uncomfortable for users. In addition, for high-density electrode arrangements, the conductive paste may flow accidentally and cause a short circuit between adjacent electrodes. Most importantly, due to the rapid dehydration of the conductive paste, it is difficult for wet electrodes to record EEG for more than a few hours.

Based on the shortcomings of the above traditional non-invasive dry electrodes and wet electrodes, some scholars put forward the concept of semi-dry electrodes. The semi-dry electrode uses the electrolyte solution of its material to establish the ion pathway between the electrode and the skin, so that it can achieve high acquisition accuracy comparable to the wet electrode and can be used as conveniently as the dry electrode. At present, many people have designed some materials for semi-dry electrodes, but these materials depend on the devices built into the electrode that can release the electrolyte, and they will crystallize due to water loss during use. Hydrogel materials are ideal materials for non-invasive semi-dry electrodes because of their high similarity to biological tissues and their versatility in electrical and mechanical aspects. However, there are still some shortcomings, for example, it is easy to lose water and easy to break, it has weak adhesion to the substrate, and safety problems under wearing conditions exist.

### SUMMARY

The purpose of the invention is to disclose a preparation method for a hydrogel electrode, the hydrogel electrode is a semi-dry double-layer hydrogel electrode, the electrode can not only record EEG signals at a resolution comparable to that of the wet electrode but also achieve continuous EEG acquisition for up to 12 hours.

To achieve the above purpose, the invention discloses a preparation method for a hydrogel electrode, the preparation method comprises the following steps:
(1) preparing a mold for a preparation of a hydrogel electrode molding;
(2) washing an electrode support with isopropanol, ethanol, and deionized water in turn and drying the electrode support thoroughly, and then treating the electrode support with a plasma surface processor;
(3) assembling and fixing the electrode support and an Ag/AgCl button to the mold;
(4) injecting a prepolymer solution of an adhesive hydrogel and a prepolymer solution of a conductive hydrogel into the mold in turn, and obtaining a hydrogel electrode with conductive and substrate-adhesion double layers after being taken out from the mold after curing.

Step (2) also comprises a functionalization step: placing the electrode support immediately in a functional solution for functionalization after using the plasma surface processor, incubating the electrode support at room temperature, cleaning, and drying;

the functional solution is any one of 3-(trimethoxysily)propyl methacrylate solution, benzophenone solution and 1,6-hexanediamine solution;

the electrode comprises the conductive hydrogel, the adhesive hydrogel, the electrode support, and the Ag/AgCl button;

one end of the conductive hydrogel penetrates the electrode support and is connected to an inner wall of the electrode support by the adhesive hydrogel;

the adhesive hydrogel is combined with a conductive layer and the electrode support through chemical bonds to avoid an influence on the use of semi-dry electrodes caused by interface delamination and friction between the hydrogel material and the support;

the Ag/AgCl button is arranged on a bottom of the electrode support and in contact with the conductive hydrogel;

a preparation method for the prepolymer solution of the conductive hydrogel comprises the following steps:

dissolving sodium alginate, acrylamide, potassium chloride, N,N,N′,N′-tetramethylethylenediamine, N,N′-methylenebisacrylamide, calcium sulfate, ammonium persulfate, glycerol, Lipidure, poly (3,4-ethylenedioxythiophene):poly (styrenesulfonate) in deionized water, preparing the prepolymer solution of conductive hydrogel by mixing uniformly after ultrasonic degassing;

a concentration of sodium alginate is 2wt %, a concentration of acrylamide is 8-16 wt %, a concentration of potassium chloride is 0.5-1.5 mol/L, a concentration of N,N,N′,N′-tetramethylethylenediamine is 0.2 % vol, a concentration of N, N'-methylenebisacrylamide is 0.066-0.330 wt %, a concentration of calcium sulfate is 0.26 wt %, a concentration of ammonium persulfate is 0.04 wt %, a concentration of glycerol is 15 % vol, a concentration of Lipidure is 5-50 % vol, and a concentration of poly (3,4-ethylenedioxythiophene):poly (styrenesulfonate) is 15 % vol.

Preferably, a preparation method for the prepolymer solution of the adhesive hydrogel comprises the following steps: dissolving sodium alginate, acrylamide, potassium chloride, N,N,N′,N′-tetramethylethylenediamine, N,N′-methylenebisacrylamide, calcium sulfate, ammonium persulfate and glycerol in deionized water, and preparing the prepolymer solution of adhesive hydrogel by mixing uniformly after ultrasonic degassing; the concentration of sodium alginate is 2 wt %, the concentration of acrylamide is 12 wt %, the concentration of potassium chloride is 0.9 wt %, the concentration of N,N,N′,N′-tetramethylethylenediamine is 0.15 % vol, the concentration of N,N′-methylenebisacrylamide is 0.03 wt %, the concentration of calcium sulfate is 0.26 wt %, the concentration of ammonium persulfate is 0.04 wt %, and the concentration of glycerol is 10 % vol.

Preferably, irradiating in an ultraviolet curing oven for 120 seconds and then placing in an oven at 50° C. for 1 hour.

Preferably, a material of the electrode support comprises any material of glass, polypropylene, rubber, nylon, and polyvinyl chloride.

Therefore, the invention adopts the preparation method for the hydrogel electrode, and the following technical effects are realized:

1. In the hydrogel electrode structure of the invention, the conductive hydrogel has a low electrode-skin contact impedance and high robustness, and can be used for a long time and repeatedly; the adhesive hydrogel can be combined with the conductive layer and the electrode support through chemical bonds to avoid the influence on the use of semi-dry electrode caused by the interface delamination and the friction between the hydrogel material and the support.

2. The invention adjusts the ratio of electrolyte content (water and potassium chloride) in the hydrogel precursor solution to enhance the ion mobility, thereby achieving high conductivity, and at the same time adjusting the balance between high conductivity and high mechanical strength of the hydrogel; glycerol and Lipidure are added to the hydrogel precursor solution to increase the moisture retention of the conductive hydrogel and prevent water loss, the monomer ratio and MBAA content are adjusted to further improve the stability of the hydrogel and determine the more comfortable mechanical strength when wearing; finally, conductive polymers, poly (3,4-ethylenedioxythiophene) (PEDOT) and poly (styrenesulfonate) (PSS) are added to the gel precursor to promote the electron conduction in the gel and the movement of ions.

3. The water retention in the hydrogel electrode of the invention is constant, and the added moisturizers are biosafe glycerol and Lipidure. Among them, glycerol is mainly dispersed in the solute of the hydrogel system, it can actively absorb water in a humid environment to achieve water absorption and moisture retention depending on the formation of hydrogen bonds between the hydroxyl groups contained in glycerol and water molecules. As a polymer moisturizer, Lipidure contains double bonds in its structure, which can polymerize under the action of free radicals in the hydrogel system, and then bind to the double network backbone of the hydrogel by chemical bonds, in addition, the hydrophobic chain in Lipidure can also form hydrogen bonds with water molecules in the hydrogel system, thereby achieving moisturizing effect. The two moisturizers are added to the hydrogel system in an appropriate ratio, which plays a synergistic role in water absorption and moisture retention without affecting the precursor of hydrogel curing. In addition, after Lipidure is dissolved in water, the polymer chain is topologically formed into an entanglement network, which increases the volume of the polymer molecule and reduces the free activity space, thereby increasing the viscosity of the system, having a certain thickening effect and promoting the adhesion of the hydrogel.

4. The measured skin contact impedance of the hydrogel electrode is comparable to that of the wet electrode (conductive paste), which is significantly lower than that of the dry electrode (metal needle electrode), the hydrogel electrode will not cause any damage to the skin under the same pressure and action time, while the metal needle electrode will cause irreversible damage to the skin.

5. In the hydrogel electrode of the invention, the adhesive layer hydrogel can well bond the electrode support and the conductive layer gel. Among them, the adhesive hydrogel can establish a chemical bond with the chemical functional groups grafted on the electrode support, at the same time, because of its similarity with the conductive hydrogel composition, the two hydrogels are seamlessly connected, and the time of firm adhesion is more than 14 days, which greatly increases the number of times the electrode can be reused.

6. The hydrogel electrode of the invention has been evaluated in human volunteers in the N170 event-related potential (ERP) test. The results show that the hydrogel electrode can capture the expected ERP waveform in the N170 test, which is similar to the waveform generated by the wet electrode. In addition, the hydrogel electrode of the invention can collect EEG for up to 12 hours and can be used for recycling (14-day test). In summary, these results indicate that the semi-dry double-layer hydrogel electrode of the invention can detect ERP in an easy-to-use manner for a long time, and it is possible to open up many applications in the real scenario of non-invasive BCI.

### BRIEF DESCRIPTION OF THE DRAWINGS

FIG. 1 is a schematic diagram of the hydrogel electrode structure of the invention;

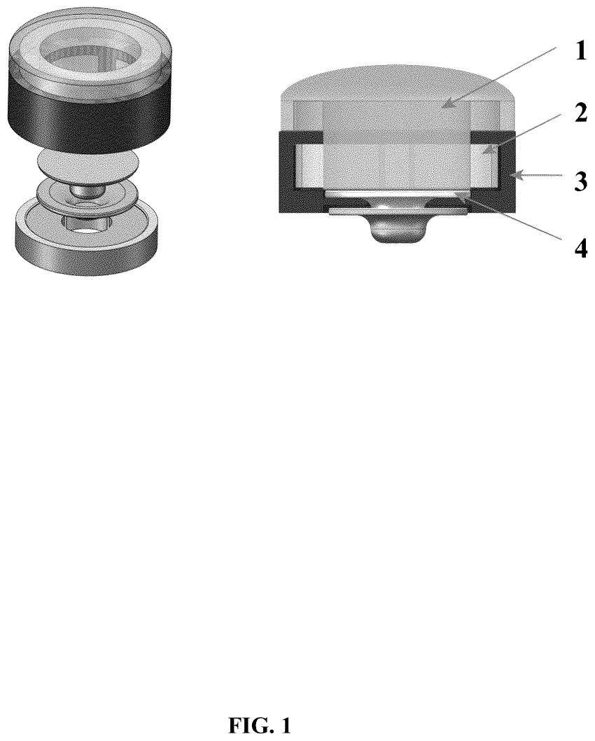

Marks in the figures: 1, conductive hydrogel, 2, adhesive hydrogel, 3, electrode support, 4, Ag/AgCl button;

FIG. 2 is a physical photo of the hydrogel electrode of the invention;

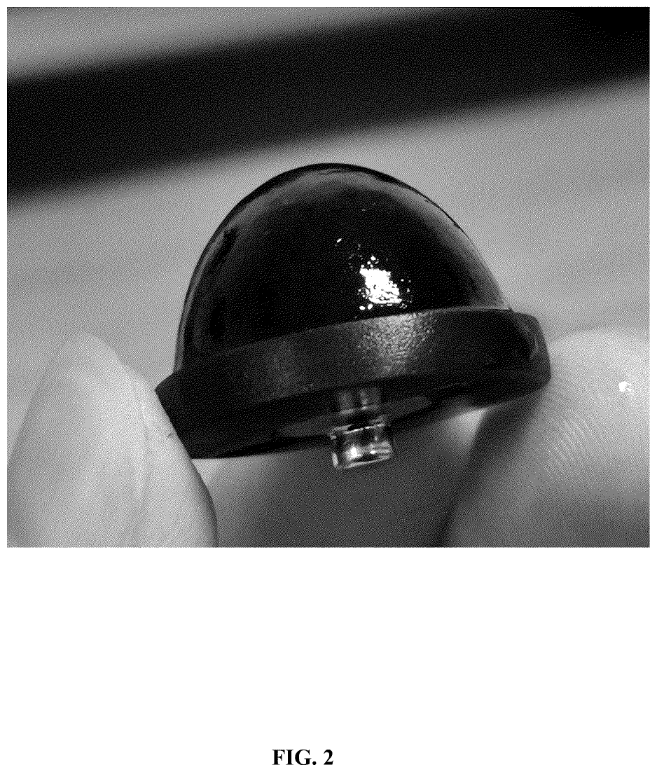

FIG. 3 is a change diagram of the hydrophilic contact angle of the electrode supports of different materials of the invention before and after plasma cleaning.

FIG. 4 is a change diagram of the hydrophilic contact angle of the electrode supports of different materials of the invention before and after plasma cleaning.

FIG. 5 is an adhesion strength diagram of the adhesive hydrogel to the electrode supports of different materials after chemical treatment with HDMA, TMSPMA, and BP, respectively.

FIG. 6 is a variation of the adhesion strength of the adhesive hydrogel to the electrode supports of different materials with time, where a is the control group, b is the BP treatment group, and c is the TMSPMA treatment group;

FIG. 7 is a moisturizing performance diagram of the conductive hydrogel of the moisturizer with different components of the invention;

FIG. 8 is a water loss comparison diagram of the conductive hydrogel with/without glycerol after 7 days of storage;

FIG. 9 is an original EEG signal waveform comparison diagram of the wet electrode and the hydrogel electrode of the invention, which are worn for 8 hours and continuously collected, a is the wet electrode and b is the hydrogel electrode;

FIG. 10 is an original EEG waveform diagram of the hydrogel electrode used continuously for 14 days (2 hours/day) in the invention;

FIG. 11 is a pig skin condition diagram of the hydrogel electrode and the dry electrode of the invention after 12 hours of continuous action on the pig skin under the same pressure.

### DETAILED DESCRIPTION OF THE EMBODIMENTS

The following embodiments can enable ordinary technicians in this field to understand the invention more comprehensively, but those embodiments do not contribute any limitations to the invention in any way.

If no special explanation is provided, the materials used in the embodiment of the invention can be obtained commercially or prepared according to the conventional method known to the technical personnel in the field.

### Embodiment 1

(1) The matching support and mold of non-invasive conductive-substrate adhesion double-layer hydrogel electrode are designed by Solidwork, and 3D printing is carried out, the electrode support is PA;

(2) The electrode support is washed with isopropanol, ethanol, and deionized water in turn and dried thoroughly, and then treated with a plasma surface processor (100 W, TS-PL 10) for 5 minutes;

(3) The electrode support and Ag/AgCl button are assembled and fixed to the mold;

(4) The adhesive hydrogel is injected:

0.2 g sodium alginate (SA), 1.2 g acrylamide (AM), 0.09 g potassium chloride (KCl), 15 μL N,N,N′,N′-tetramethylethylenediamine (TEMED), 0.003 g N,N′-methylenebisacrylamide (MBAA), 0.26 g calcium sulfate (CaSO4), 0.004 g ammonium persulfate (APS), 1 ml glycerol are dissolved in 10 ml deionized water, mixed uniformly after ultrasonic degassing, and the prepolymer solution of the adhesive hydrogel is prepared by mixing uniformly after ultrasonic degassing, and the prepolymer solution of the adhesive hydrogel is injected into the corresponding position of the mold and the electrode support.

(5) The conductive hydrogel is injected:

0.2 g sodium alginate (SA), 1.2 g acrylamide (AM), 0.7455 g potassium chloride (KCl), 20 μL N,N,N′,N′-tetramethylethylenediamine (TEMED), 0.0066 g N,N′-methylenebisacrylamide (MBAA), 0.026 g calcium sulfate (CaSO4), 0.004 g ammonium persulfate (APS), 1.5 ml glycerol, 1.5 ml Lipidure, 1.5 ml poly (3,4-ethylenedioxythiophene): poly (styrenesulfonate) (PEDOT:PSS) are dissolved in 10 ml deionized water, the prepolymer solution of the conductive hydrogel is prepared by mixing uniformly after ultrasonic degassing, and the prepolymer solution of the conductive hydrogel is injected into the corresponding mold.

(6) The hydrogel electrode is obtained by irradiating in an ultraviolet curing oven (UVHX 300-400,100 % power) for 120 seconds and then placing in an oven at 50° C. for 1 hour.

### Embodiments 2-5

The preparation method is similar to that of Embodiment 1, the difference is that the electrode supports are glass, rubber, PVC, and PP, respectively.

### Embodiment 6

The preparation method is similar to that of Embodiment 1. The difference is that in Step (2), the electrode support is successively washed with isopropanol, ethanol, and deionized water and thoroughly dried, and then treated with a plasma surface processor (100 W, TS-PL 10) for 5 minutes, and then the support is immediately placed in a functional solution, incubated at room temperature, cleaned and dried, and the functionalized substrate is stored under low humidity conditions before being used for the experiment;

the functionalization is carried out in the following ways:

3-(trimethoxysily)propyl methacrylate (TMSPMA) solution: 100 ml deionized water, 10 μl acetic acid, and 2 g TMSPMA are incubated at room temperature for 6 h, the electrode support is washed with ethanol, and completely dried, the functionalized electrode support is stored under low humidity conditions before being used in the experiment.

### Embodiments 7-10

The preparation process is similar to that of Embodiment 6, the difference is that the electrode supports are glass, rubber, PVC, and PP, respectively.

### Embodiment 11

The preparation method is similar to that of Embodiment 1, the difference is that in Step (2), the electrode support is successively washed with isopropanol, ethanol, and deionized water and thoroughly dried, and then treated with a plasma surface processor (100 W, TS-PL 10) for 5 minutes, and then the electrode support is immediately placed in a functional solution, incubated at room temperature, cleaned and dried, and the functionalized substrate is stored under low humidity conditions before being used for the experiment; the functionalization is carried out in the following ways:

Benzophenone (BP) solution: 90 ml deionized water, 10 ml ethanol, and 2.5 g BP are incubated at room temperature for 3 h, the electrode support is washed with methanol and completely dried, the functionalized electrode support is stored under low humidity conditions before being used for the experiment.

### Embodiments 12-15

The preparation process is similar to that of Embodiment 11. The difference is that the electrode supports are glass, rubber, PVC, and PP, respectively.

### Embodiment 16

The preparation method is similar to that of Embodiment 1, the difference is that in Step (2), the electrode support is successively washed with isopropanol, ethanol, and deionized water and thoroughly dried, and then treated with a plasma surface processor (100 W, TS-PL 10) for 5 minutes, and then the electrode support is immediately placed in a functional solution, incubated at room temperature, cleaned and dried, and the functionalized substrate is stored under low humidity conditions before being used for the experiment;

the functionalization is carried out in the following ways:

1,6-hexanediamine (HDMA) solution: 90 ml deionized water and 10 ml HDMA are incubated at room temperature for 24 h, the electrode support is washed with isopropanol and completely dried, the functionalized electrode support is stored under low humidity conditions before being used for the experiment.

### Embodiments 17-20

The preparation process is similar to that of Embodiment 16, the difference is that the electrode supports are glass, rubber, PVC, and PP, respectively.

Analysis of test results:

### 1. Adhesion Performance

The bonding between the adhesive hydrogel and several different electrode supports is studied, and the adhesion performances are tested, the electrode supports comprise glass, rubber, PA, PVC, and PP.

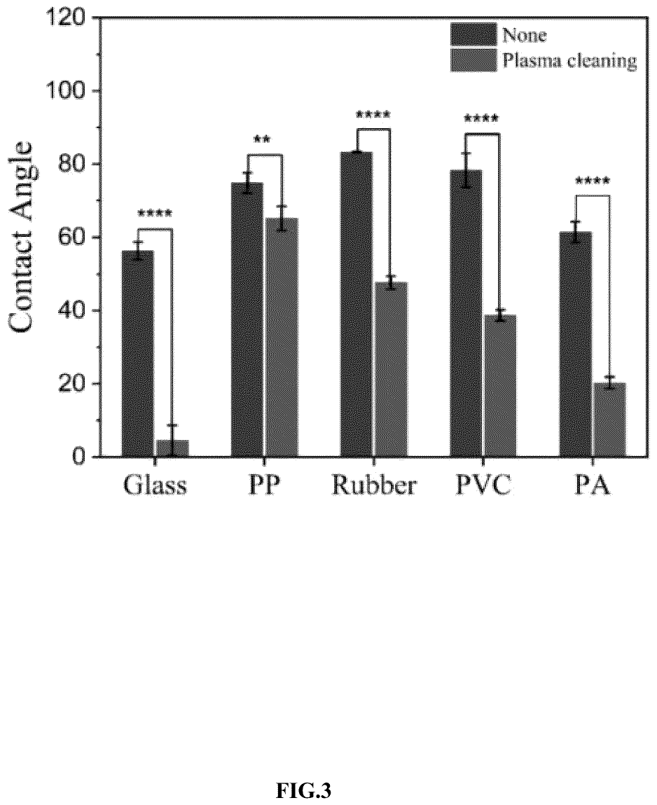

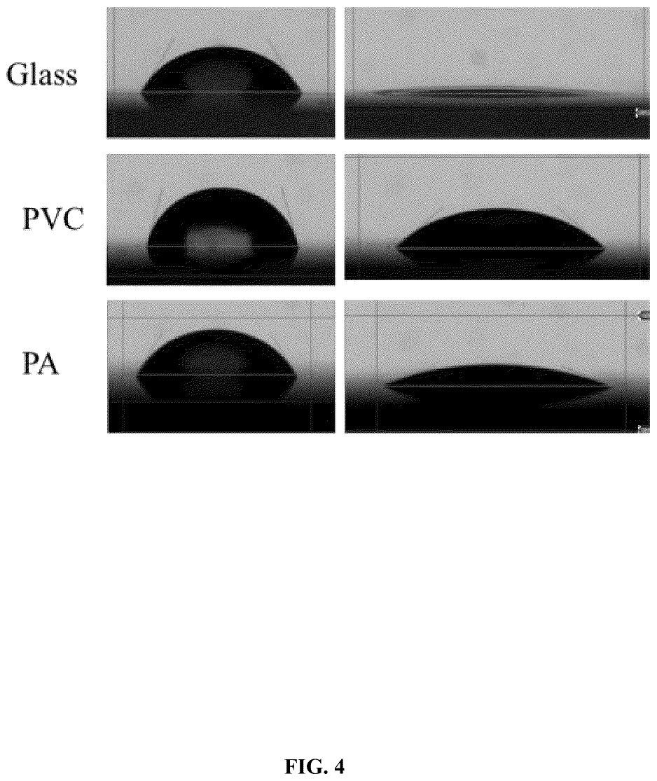

After pre-cleaning and oxygen plasma cleaning of the electrode support, the surfaces of these substrates become rougher, and hydroxyl groups (hydrophilic angle changes) that are easy to react with the functional solution are obtained on the surface, therefore it has better adhesion to the hydrogel (FIGS. 3, 4).

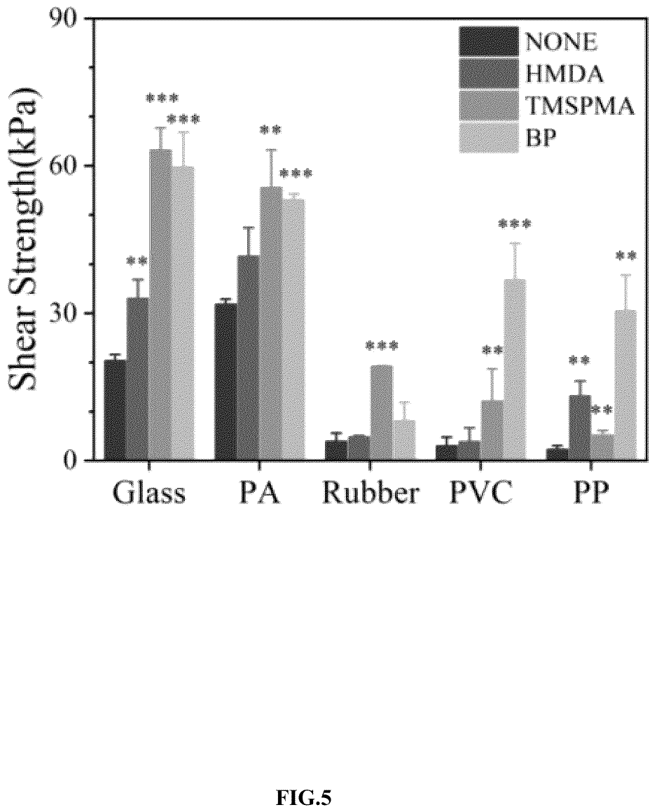

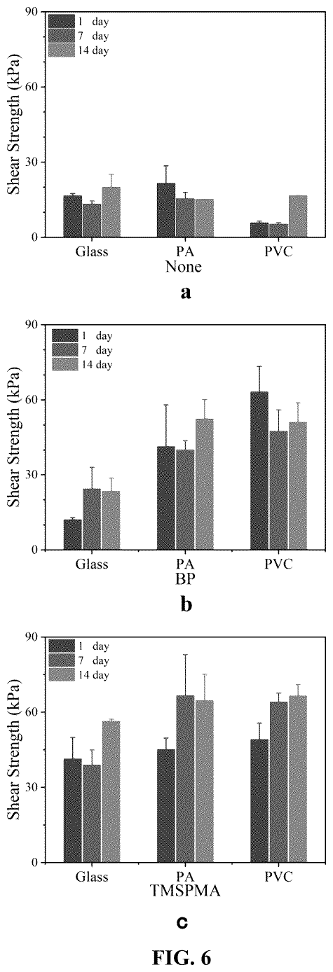

In order to test the bonding toughness of the adhesive hydrogel and the treated electrode supports, we performed a lap shear test, and the results are shown in FIG. 5. It can be seen that after different chemical treatments on the electrode supports, the adhesion performances of the adhesive hydrogel are significantly improved compared with the untreated substrate. The long-term stability of adhesive hydrogels and electrode supports is crucial for the sustainable use of hydrogel electrodes. Glass, PA, and PVC with good adhesion to the adhesive hydrogel are selected, and TMSPMA and BP are used as chemical treatments. The shear strength between the adhesive hydrogel and the electrode support is measured on the 1st, 7th, and 14th days after adhesion. The results are shown in FIG. 6 (a-c). The shear strength of the adhesive hydrogel does not decrease with time, indicating that the adhesion of the adhesive hydrogel to the electrode support had long-term stability. Due to the similar composition of the two hydrogels, the adhesion between the adhesive hydrogel and the conductive hydrogel has no obvious layered interface after curing, and the two hydrogels are perfectly integrated.

### 2, Moisture Retentiveness

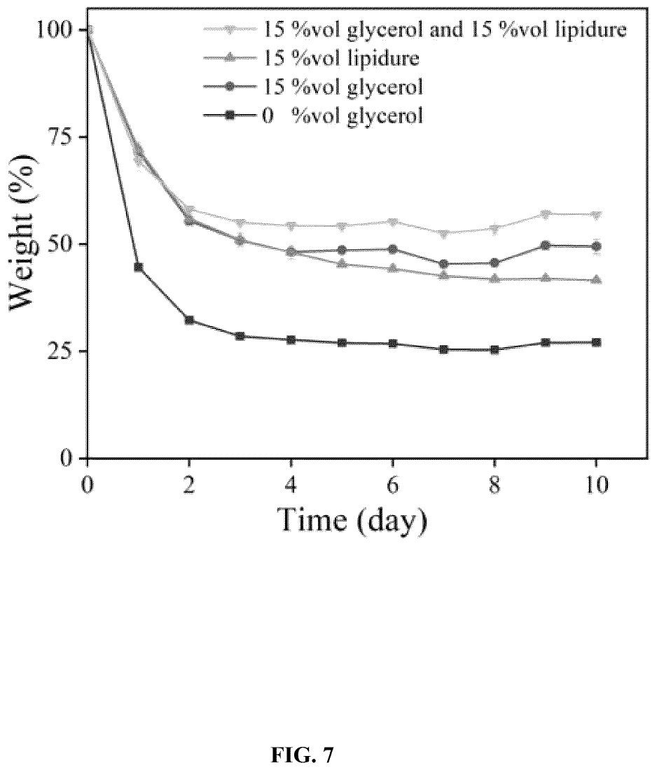

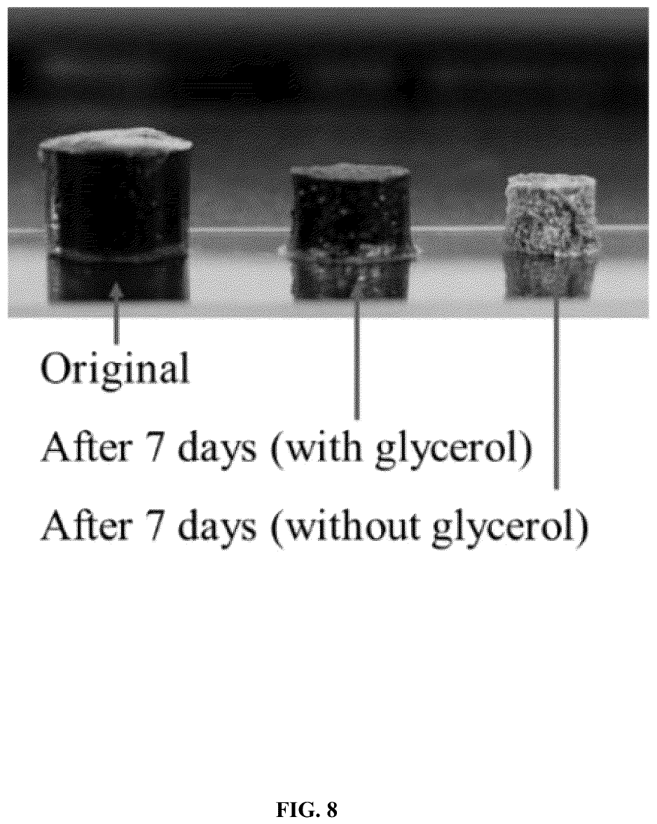

The effects of glycerol, Lipidure, glycerol, and Lipidure, glycerol, and Lipidure on the moisture retention of conductive hydrogels are investigated by moisture retentiveness test. It is found that the weight of the conductive hydrogel containing glycerol and Lipidure moisturizing in Embodiment 1 decreases after fresh curing, and becomes stable after 3 days of exposure to air, and its water content can be maintained at about 60%. On the contrary, the hydrogels without humectants shrink to about 25% of their mass after 3 days of exposure to air (FIG. 7). It can be seen from FIG. 8 that the hydrogel containing glycerol can maintain its normal appearance after 7 days, while the hydrogel without humectant is dehydrated and dried.

### 3. EEG Signal Acquisition Results

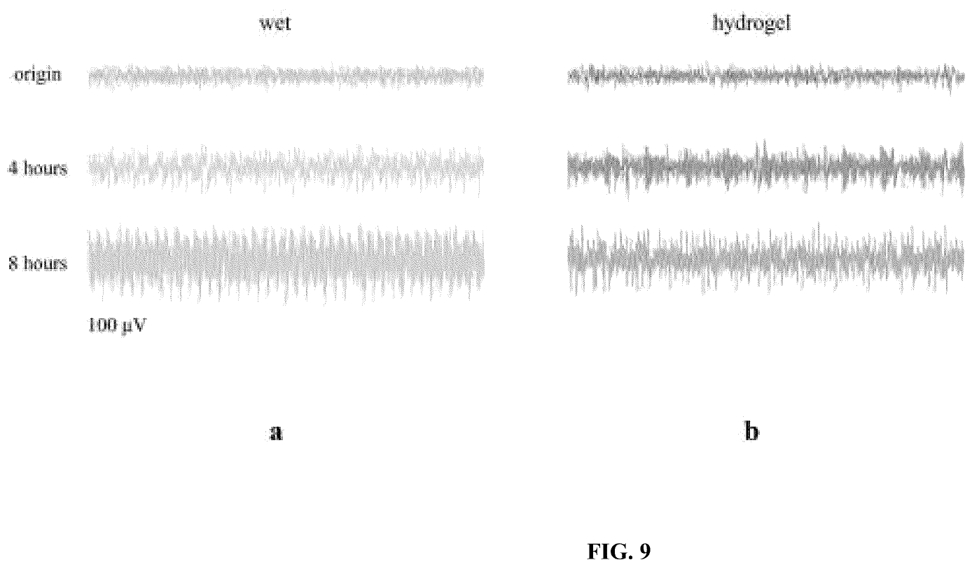

Event-related potential (EPR) is effectively used to evaluate brain functions by measuring EEG. In terms of signal acquisition, ERP provides a practical method to determine the quality of EEG collected by non-invasive EEG electrodes. Here, we performed N170 tests on human volunteers by using the hydrogel electrodes prepared from wet, dry, and Embodiment 6 respectively in a sound-proof environment. In the N170 test, the electrode position Oz is selected for analysis. FIG. 9 shows the original EEG signals collected by the wet electrode and the hydrogel electrode, it is found that the hydrogel electrode and the wet electrode have similar original signal forms.

### 4. Long-Term Performance of the Electrode

The comfort of the electrode is also an important factor to be considered in practical use. For the hydrogel electrode, the subjects do not report any discomfort, and no hair or skin residues are found on the electrode after long-term use. In addition, the hydrogel electrode should be easy to install on the EEG cap and have the characteristics of convenient use. The use of wet electrodes requires the injection of conductive paste, which will cause discomfort to the subject's scalp. For the needle-shaped dry electrode, the subject's head will experience discomfort and pain.

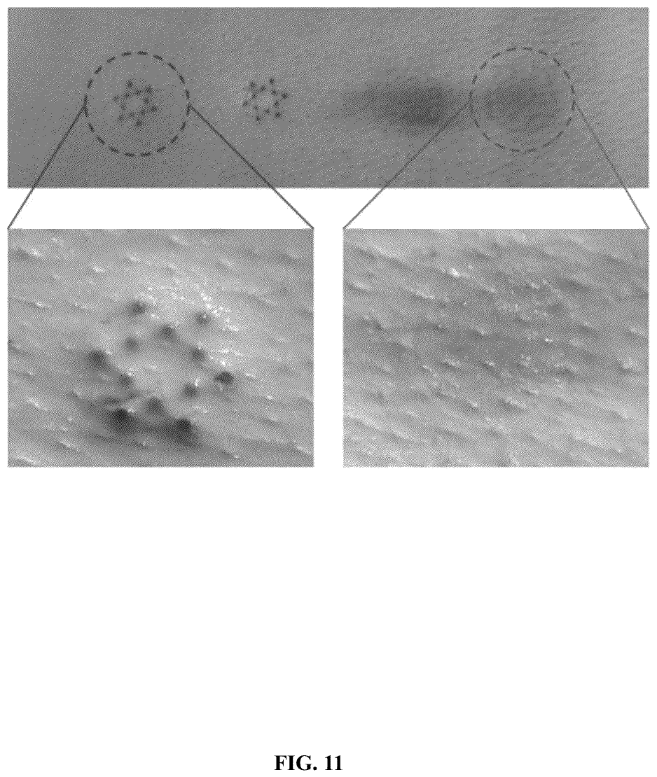

We performed long-term pressure tests on animal skin (pig skin) using hydrogel electrodes and dry electrodes prepared by Embodiment 6. It was found that the hydrogel electrode did not cause any damage to the skin after 12 hours of application, while the metal needle electrode caused considerable damage to the skin (FIG. 11).

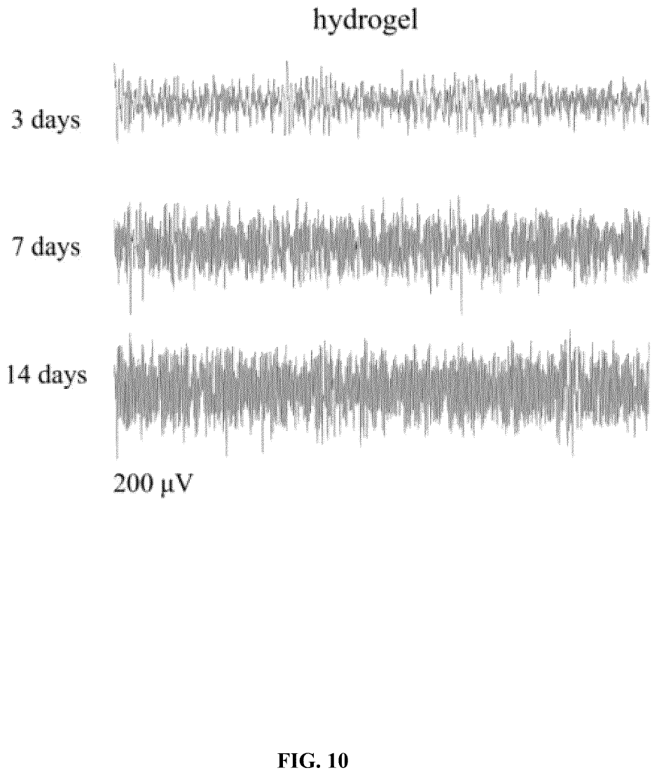

Whether the hydrogel electrode can be reused for a long time determines whether it can be truly applied to real-world scenarios. Therefore, we used the EEG cap to test the long-term stability of the hydrogel electrode. In the long-term N170 test, we recorded the EEG signals of human subjects for 8 consecutive hours using a conductive hydrogel electrode (FIG. 9). During this period, the waveform recorded by the hydrogel electrode is very similar to that of the wet electrode, and the noise level presented by the hydrogel electrode is much lower than that of the wet electrode after wearing for 8 hours. After 8 hours, the impedance becomes infinite due to the drying of the conductive paste. At the same time, the real-time impedance of the hydrogel electrode remains below 150 kΩ within 12 hours. In addition, in order to test whether the hydrogel electrode can be recycled, we conducted an N170 test within 14 days of reusing the hydrogel electrode, it was found that the hydrogel electrode can still work normally even after 14 days of continuous use, and the recorded EEG signal is shown in FIG. 10.

The above embodiments are only the preferred embodiments of the invention, which are not the limitations of the implementation methods. The scope of protection of the invention shall be subject to the scope limited by the claims. Based on the above description, other different forms of changes or amendments can be made, the obvious changes or variations therefrom are still within the scope of protection of the invention.

What is claimed is:

1. A preparation method for a hydrogel electrode, the preparation method comprises the following steps:
(1) preparing a mold for a preparation of a hydrogel electrode molding;
(2) washing an electrode support with isopropanol, ethanol, and deionized water in turn and drying the electrode support thoroughly, and then treating the electrode support with a plasma surface processor;
(3) assembling and fixing the electrode support and an Ag/AgCl button to the mold;
(4) injecting a prepolymer solution of an adhesive hydrogel and a prepolymer solution of a conductive hydrogel into the mold in turn, and obtaining a hydrogel electrode with conductive and substrate-adhesion double layers after being taken out from the mold after curing.
Step (2) also comprises a functionalization step: placing the electrode support immediately in a functional solution for functionalization after using the plasma surface processor, incubating the electrode support at room temperature, cleaning, and drying;
the functional solution is any one of 3-(trimethoxysily)propyl methacrylate solution, benzophenone solution and 1,6-hexanediamine solution;
the electrode comprises the conductive hydrogel, the adhesive hydrogel, the electrode support, and the Ag/AgCl button;
one end of the conductive hydrogel penetrates the electrode support and is connected to an inner wall of the electrode support by the adhesive hydrogel;
the adhesive hydrogel is combined with a conductive layer and the electrode support through chemical bonds to avoid an influence on a use of semi-dry electrodes caused by interface delamination and friction between the hydrogel material and the support;
the Ag/AgCl button is arranged on a bottom of the electrode support and in contact with the conductive hydrogel;
a preparation method for the prepolymer solution of the conductive hydrogel comprises the following steps:
dissolving sodium alginate, acrylamide, potassium chloride, N,N,N′,N′-tetramethylethylenediamine, N,N′-methylenebisacrylamide, calcium sulfate, ammonium persulfate, glycerol, Lipidure, poly (3,4-ethylenedioxythiophene):poly (styrenesulfonate) in deionized water, preparing the prepolymer solution of conductive hydrogel by mixing uniformly after ultrasonic degassing;
a concentration of sodium alginate is 2 wt %, a concentration of acrylamide is 8-16 wt %, a concentration of potassium chloride is 0.5-1.5 mol/L, a concentration of N,N,N′,N′-tetramethylethylenediamine is 0.2 % vol, a concentration of N,N′-methylenebisacrylamide is 0.066-0.330 wt %, a concentration of calcium sulfate is 0.26 wt %, a concentration of ammonium persulfate is 0.04 wt %, a concentration of glycerol is 15 % vol, a concentration of Lipidure is 5-50 % vol, and a concentration of poly (3,4-ethylenedioxythiophene):poly (styrenesulfonate) is 15 % vol.

2. The preparation method according to claim 1, wherein a preparation method for a prepolymer solution of the adhesive hydrogel comprises the following steps: dissolving sodium alginate, acrylamide, potassium chloride, N,N,N′,N′-tetramethylethylenediamine, N,N′-methylenebisacrylamide, calcium sulfate, ammonium persulfate and glycerol in deionized water, and preparing the prepolymer solution of adhesive hydrogel by mixing uniformly after ultrasonic degassing;
the concentration of sodium alginate is 2 wt %, the concentration of acrylamide is 12 wt %, the concentration of potassium chloride is 0.9 wt %, the concentration of N,N,N′,N′-tetramethylethylenediamine is 0.15 % vol, the concentration of N,N′-methylenebisacrylamide is 0.03 wt %, the concentration of calcium sulfate is 0.26 wt %, the concentration of ammonium persulfate is 0.04 wt %, and the concentration of glycerol is 10 % vol.

3. The preparation method according to claim 1, wherein irradiating in an ultraviolet curing oven for 120 seconds and then placing in an oven at 50 ° C. for 1 hour.

4. The preparation method according to claim 1, wherein a material of the electrode support comprises any of glass, polypropylene, rubber, nylon, and polyvinyl chloride.
